(19) United States
(12) Patent Application Publication
(10) Pub. No.: US 2011/0046180 A1
(43) Pub. Date: Feb. 24, 2011

(54) N-ARYL-N-PIPERIDIN-4-YL-PROPIONAMIDE DERIVATIVES AND THEIR USE AS OPIOID RECEPTOR LIGANDS

(75) Inventors: Dan Peters, Malmo (SE); Birgitte L. Eriksen, Farum (DK); Gordon Munro, Kobenhavn NV (DK); Elsebet Østergaard Nielsen, Kobenhavn K (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/809,156

(22) PCT Filed: Dec. 18, 2008
(86) PCT No.: PCT/EP2008/067849

§ 371 Date: Oct. 25, 2010

Related U.S. Application Data
(60) Provisional application No. 61/015,211, filed on Dec. 20, 2007.

(30) Foreign Application Priority Data
Dec. 19, 2007 (DK) PA 2007 01822

(51) Int. Cl. A61K 31/445; C07D 211/56; A61P 17/00; A61P 25/36; A61P 25/00; A61P 25/04; A61P 25/32

(52) U.S. Cl. 514/329; 546/224

(57) ABSTRACT

This invention relates to novel N-aryl-N-piperidin-4-yl-propionamide derivatives useful as opioid receptor ligands.

In other aspects the invention relates to the use of these compounds in a method for therapy, such as for the treatment of pain, and to pharmaceutical compositions comprising the compounds of the invention.

### TECHNICAL FIELD

This invention relates to novel N-aryl-N-piperidin-4-yl-propionamide derivatives useful as opioid receptor ligands. More specifically, the invention provides compounds useful as μ opioid receptor ligands.

In other aspects the invention relates to the use of these compounds in a method for therapy, such as for the treatment of pain, and to pharmaceutical compositions comprising the compounds of the invention.

### BACKGROUND ART

Numerous classes of opioid receptors exist. These classes differ in their affinity for various opioid ligands and in their cellular and organ distribution. Moreover, although the different classes are believed to serve different physiological functions, there is a substantial overlap of function, as well as distribution. Three different types of opioid receptors have been identified, the mu (μ), delta (δ) and kappa (κ) opioid receptor. These three opioid receptor types are the sites of action of opioid ligands producing analgesic effects. However, the type of pain inhibited and the secondary functions vary with each receptor type. The μ receptor is generally regarded as primarily associated with pain relief, and drug or other chemical dependence, such as addiction or alcoholism. The δ receptor appears to deal with behavioural effects, although the δ and the κ receptors may also mediate analgesia.

Each opioid receptor, when coupled with an opiate, causes a specific biological response unique to that type of receptor. When an opiate activates more than one receptor, the biological response for each receptor is affected, thereby producing side effects. The less specific and selective an opiate may be, the greater the chance of causing increased side effect by the administration of the opiate.

Whereas morphine, which is a strong opioid analgetic agent shows effectiveness against strong pain by acting on the μ opioid receptor (agonist activity), there is a problem that its side effects such as nausea and neurologic manifestation including hallucination and derangement. Moreover, morphine forms psychological dependence, causing serious problems. Other side effects reported are respiratory depression, tolerance, physical dependence capacity, and precipitated withdrawal syndrome, caused by non-specific interactions with central nervous receptors.

### SUMMARY OF THE INVENTION

It is an object of the invention to provide novel compounds which act on opiate receptors.

A further object of the invention is the provision of compounds that substantially avoid the unwanted side effects associated with conventional peripherally acting analgesics.

It is a further object to provide compounds that bind selectively to the μ opioid receptor.

A still further object is the provision of compounds which optionally—in addition to the μ opioid receptor activity—show activity as monoamine neurotransmitter re-uptake inhibitors.

In its first aspect, the invention provides a compound of Formula I,

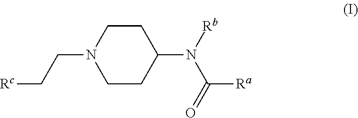

any of its stereoisomers or any mixture of its stereoisomers, or a pharmaceutically acceptable salt thereof; wherein R^{a}, R^{b} and R^{c} are as defined below.

In its second aspect, the invention provides a pharmaceutical composition, comprising a therapeutically effective amount of a compound of the invention, any of its stereoisomers or any mixture of its stereoisomers, or a pharmaceutically acceptable salt thereof, together with at least one pharmaceutically acceptable carrier, excipient or diluent.

In a further aspect, the invention provides the use of a compound of the invention, any of its stereoisomers or any mixture of its stereoisomers, or a pharmaceutically acceptable salt thereof, for the manufacture of a pharmaceutical composition for the treatment, prevention or alleviation of a disease or a disorder or a condition of a mammal, including a human, which disease, disorder or condition is responsive to modulation of the opioid receptor.

In a still further aspect, the invention relates to a method for treatment, prevention or alleviation of a disease or a disorder or a condition of a living animal body, including a human, which disorder, disease or condition is responsive to responsive to modulation of the opioid receptor, which method comprises the step of administering to such a living animal body in need thereof a therapeutically effective amount of a compound of the invention, any of its stereoisomers or any mixture of its stereoisomers, or a pharmaceutically acceptable salt thereof.

Other objects of the invention will be apparent to the person skilled in the art from the following detailed description and examples.

### DETAILED DISCLOSURE OF THE INVENTION

### N-aryl-N-piperidin-4-yl-propionamide Derivatives

In its first aspect, the invention provides a compound of Formula I,

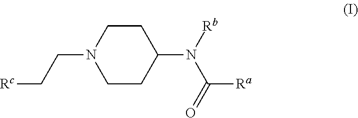

any of its stereoisomers or any mixture of its stereoisomers, or a pharmaceutically acceptable salt thereof;
wherein
R^{a} represents hydrogen or alkyl;

which alkyl is optionally substituted with one or more substituents independently selected from the group consisting of:
halo, trifluoromethyl, trifluoromethoxy, cyano and alkoxy;
   R^{b} and R^{c} independent of each other represent an aryl group;

which aryl group is substituted with one or more substituents independently selected from the group consisting of:
halo, trifluoromethyl, trifluoromethoxy, cyano and alkoxy.

In one embodiment of the compound of formula I, R^{a} represents alkyl. In a special embodiment, R^{a} represents ethyl.

In a further embodiment of the compound of formula I, R^{b} represents a substituted phenyl. In a special embodiment, R^{b} represents a dihalosubstituted phenyl, such as dichlorophenyl, in particular 3,4-dichlorophenyl, or difluorophenyl, in particular 3,4-difluorophenyl. In a further embodiment, R^{b} represents a halosubstituted phenyl, such as chlorophenyl, in particular 4-chlorophenyl. In a still further embodiment, R^{b} represents a phenyl substituted with halo and alkoxy, e.g. chloro and methoxy. In a special embodiment, R^{b} represents 4-chloro-3-methoxy-phenyl.

In a still further embodiment of the compound of formula I, R^{c} represents a substituted phenyl. In a special embodiment, R^{c} represents halophenyl, such as fluorophenyl, in particular 3-fluorophenyl or 4-fluorophenyl. In a further embodiment, R^{c} represents alkoxyphenyl, such as methoxyphenyl, in particular 4-methoxyphenyl.

In a special embodiment the compound of the invention is
N-(3,4-Difluoro-phenyl)-N-{1-[2-(4-fluoro-phenyl)-ethyl]-piperidin-4-yl}-propionamide;
N-(3,4-Difluoro-phenyl)-N-{1-[2-(4-methoxy-phenyl)-ethyl]-piperidin-4-yl}-propionamide;
N-(3,4-Difluoro-phenyl)-N-{1-[2-(3-fluoro-phenyl)-ethyl]-piperidin-4-yl}-propionamide;
N-(4-Chloro-phenyl)-N-{1-[2-(4-methoxy-phenyl)-ethyl]-piperidin-4-yl}-propionamide;
N-(4-Chloro-phenyl)-N-{1-[2-(4-fluoro-phenyl)-ethyl]-piperidin-4-yl}-propionamide;
N-(4-Chloro-phenyl)-N-{1-[2-(3-fluoro-phenyl)-ethyl]-piperidin-4-yl}-propionamide;
N-(3,4-Dichloro-phenyl)-N-{1-[2-(4-methoxy-phenyl)-ethyl]-piperidin-4-yl}-propionamide;
N-(3,4-Dichloro-phenyl)-N-{1-[2-(3-fluoro-phenyl)-ethyl]-piperidin-4-yl}-propionamide;
N-(3,4-Dichloro-phenyl)-N-{1-[2-(4-fluoro-phenyl)-ethyl]-piperidin-4-yl}-propionamide;
N-(4-Chloro-3-methoxy-phenyl)-N-{1-[2-(4-fluoro-phenyl)-ethyl]-piperidin-4-yl}-propionamide;
N-(4-Chloro-3-methoxy-phenyl)-N-{1-[2-(4-methoxy-phenyl)-ethyl]-piperidin-4-yl}-propionamide;
N-(4-Chloro-3-methoxy-phenyl)-N-{1-[2-(3-fluoro-phenyl)-ethyl]-piperidin-4-yl}-propionamide;
   or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contains of from one to six carbon atoms (C₁₋₆-alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

Alkoxy is —O-alkyl, wherein alkyl is as defined above.

In the context of this invention an aryl group designates a carbocyclic aromatic ring system such as phenyl, naphthyl (1-naphthyl or 2-naphthyl) or fluorenyl.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound of the invention include examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the compounds of the present invention may exist in different stereoisomeric forms—including enantiomers, diastereomers or cis-trans-isomers.

The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid—by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

The chemical compounds of the present invention may also be resolved by the formation of diastereomeric amides by reaction of the chemical compounds of the present invention with an optically active carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radio-nuclide as a label. Positron emitting radionuclides are all candidates for usage. In the context of this invention the radionuclide is preferably selected from ²H (deuterium), ³H (tritium), ¹¹C, ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Preparation

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

Compounds of the invention may be tested for their ability to bind to the μ, δ, and κ opioid receptors, e.g. such as described by Simonin F et al [Simonin F et al, Mol. Pharmacol., 46(6), 1015-21, 1994], Simonin F et al [Simonin F et al, Proc. Natl. Acad. Sci. USA, 92(15), 7006-10, 1995], and Wang J B et al [Wang J B et al, FEBS Lett., 348(1), 75-9, 1994].

Compounds that bind to opiate receptors, in particular the μ receptor, are likely to be useful in the treatment of pain, postoperative pain, chronic pain (such as cancer pain and neuropathic pain), pain during labour and delivery, migraine, drug addiction (such as heroin addiction and cocaine addiction), and alcoholism.

Furthermore, compounds that bind to opiate receptors are also likely to be useful in the treatment of irritable bowel syndrome, constipation, nausea, vomiting, and pruritic dermatoses (itching), such as allergic dermatitis and atopy. Compounds that bind to opiate receptors have also been indicated in the treatment of eating disorders, opiate overdoses, depression, smoking, sexual dysfunction, shock, stroke, spinal damage, head trauma, diarrhoea, urinary incontinence and inflammatory reactions.

Thus in further aspect, the compounds of the invention are considered useful for the treatment, prevention or alleviation of a disease, disorder or condition responsive to modulation of the opioid receptors, in particular the μ opioid receptor.

In a special embodiment, the compounds of the invention are considered useful for the treatment, prevention or alleviation of pain, postoperative pain, chronic pain, cancer pain, neuropathic pain, pain during labour and delivery, migraine, drug addiction, heroin addiction, cocaine addiction, alcoholism, irritable bowel syndrome, constipation, nausea, vomiting, pruritic dermatoses, allergic dermatitis, atopy, eating disorders, opiate overdoses, depression, smoking, sexual dysfunction, shock, stroke, spinal damage, head trauma, diarrhoea, urinary incontinence and inflammatory reactions.

In a further embodiment, the compounds of the invention are considered particularly useful for the treatment, prevention or alleviation of pain, postoperative pain, chronic pain, migraine, drug addiction, alcoholism, and irritable bowel syndrome.

In a still further embodiment, the compounds of the invention also show activity as monoamine neurotransmitter re-uptake inhibitors. The compounds of the invention may be tested for their ability to inhibit reuptake of the monoamines dopamine, noradrenaline and serotonin in synaptosomes e.g. such as described in WO 97/30997.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of the chemical compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to modulation of the opioid receptor, and which method comprises administering to such a living animal body, including a human, in need thereof an effective amount of a compound of the invention, any of its isomers or any mixture of its isomers, or a pharmaceutically acceptable salt thereof.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge. When administered in combination with compounds known in the art for treatment of the diseases, the dosage regimen may be reduced.

### EXAMPLES

The invention is further illustrated with reference to the following examples, which are not intended to be in any way limiting to the scope of the invention as claimed.

### Example 1

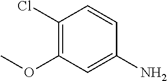

### 4-Chloro-3-methoxy-phenylamine

Ammonium chloride (14.3 g, 267 mmol) and iron powder (325 mesh, 14.9 g, 267 mmol) were dissolved in water (100 mL). A solution of 2-chloro-5-nitroanisole (10 g, 53.3 mmol) in tetrahydrofuran (50 mL) and methanol (50 mL) was added. The mixture was stirred at 80° C. overnight.

The reaction mixture was cooled to room temperature and filtrated. The filtrate was extracted with ethyl acetate. The organic phases were washed with brine, dried over sodium sulphate filtrated and concentrated in vacuo to give 4-chloro-3-methoxy-phenylamine 6.9 g (82%) as a grey solid.

### Example 2

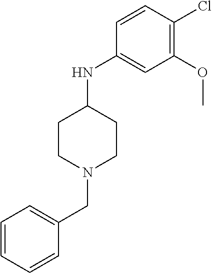

### (1-Benzyl-piperidin-4-yl)-(4-chloro-3-methoxy-phenyl)-amine

N-Benzylpiperidone (7.9 mL, 43.8 mmol), 4-chloro-3-methoxy-phenylamine (6.9 g, 43.8 mmol) and sodium sulphate (31.1 g, 218 mmol) were suspended in dichloromethane (250 mL). Sodium triacetoxyborohydride (11.4 g, 52.5 mmol) was added and the reaction mixture was stirred at room temperature overnight. Aqueous sodium hydrogencarbonate was added followed by stirring for 30 min.

The mixture was extracted with dichloromethane and the combined organic phases were washed with brine, dried over sodium sulphate, filtrated and concentrated in vacuo. (1-Benzyl-piperidin-4-yl)-(4-chloro-3-methoxy-phenyl)-amine was precipitated from ethyl acetate/heptane 6.7 g (46%) as a white solid.

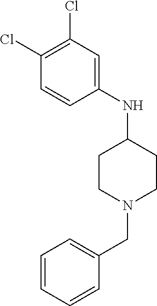

### (1-Benzyl-piperidin-4-yl)-(3,4-dichloro-phenyl)-amine

Was prepared according to Example 2 from N-benzyl-4-piperidone and 3,4-dichloroaniline.

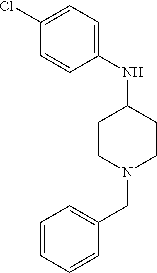

### (1-Benzyl-piperidin-4-yl)-(4-chloro-phenyl)-amine

Was prepared according to Example 2 from N-benzyl-4-piperidone and 4-chloroaniline.

### Example 3

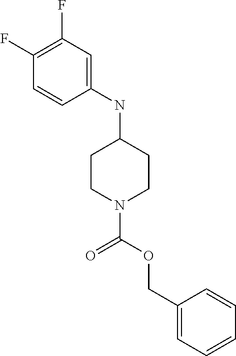

### 4-(3,4-Difluoro-phenylamino)-piperidine-1-carboxylic acid benzyl ester

Benzyl 4-oxo-1-piperidinecarboxylate (15.0 g, 64.3 mmol), 3,4-difluoroaniline (8.30 g, 64.3 mmol) and sodium sulphate (45.7 g, 321 mmol) were dissolved in 1,2-dichloroethane. Sodium triacetoxyborohydride (16.4 g, 77.2 mmol) was added and the reaction mixture was stirred at room temperature over night. Sodium hydrogencarbonate was added followed by extraction with dichloromethane. The combined organic phases were washed with water and brine, dried over sodium sulphate, filtrated and concentrated in vacuo to give 4-(3,4-difluoro-phenylamino)-piperidine-1-carboxylic acid benzyl ester (23.3 g) as an yellow oil. The material was used without further purification.

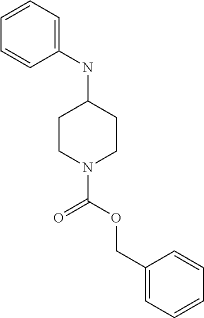

### 4-Phenylamino-piperidine-1-carboxylic acid benzyl ester

Was prepared according to Example 3 from benzyl 4-oxo-1-piperidinecarboxylate and aniline.

### Example 4

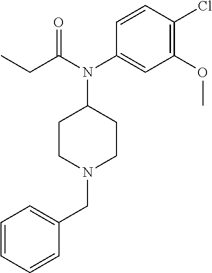

### N-(1-Benzyl-piperidin-4-yl)-N-(4-chloro-3-methoxy-phenyl)-propionamide

(1-Benzyl-piperidin-4-yl)-(4-chloro-3-methoxy-phenyl)-amine (6.7 g, 20.3 mmol) was dissolved in toluene (150 mL). Propionic anhydride (5.2 mL, 40.5 mmol) was added and the reaction mixture was heated to reflux overnight. The mixture was poured into sodium hydroxide (1 M, 150 mL) and stirred for 30 min. The organic layer was washed with water until pH 7, washed with brine, dried over sodium sulphate, filtrated and concentrated in vacuo to give N-(1-benzyl-piperidin-4-yl)-N-(4-chloro-3-methoxy-phenyl)-propionamide 6.0 g (76%).

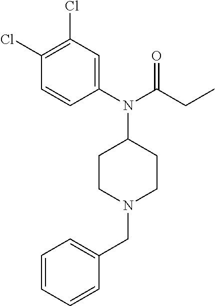

### N-(1-Benzyl-piperidin-4-yl)-N-(3,4-dichloro-phenyl)-propionamide

Was prepared according to Example 4 from (1-benzyl-piperidin-4-yl)-(3,4-dichloro-phenyl)-amine and propionic anhydride.

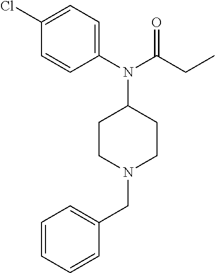

### N-(1-Benzyl-piperidin-4-yl)-N-(4-chloro-phenyl)-propionamide

Was prepared according to Example 4 from (1-benzyl-piperidin-4-yl)-(4-chloro-phenyl)-amine and propionic anhydride.

### Example 5

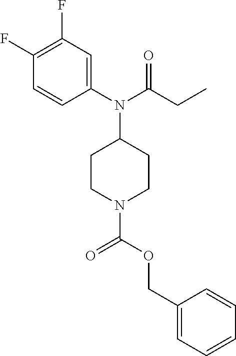

### 4-[(3,4-Difluoro-phenyl)-propionyl-amino]-piperidine-1-carboxylic acid benzyl ester

4-(3,4-Difluoro-phenylamino)-piperidine-1-carboxylic acid benzyl ester (23.3 g, 67.3 mmol) and triethylamine (19.1 mL, 134.5 mmol) were dissolved in dichloromethane (350 mL). Propionyl chloride (8.98 mL, 100.9 mmol) was added dropwise and the reaction mixture was stirred at room temperature for 3 hours. Additional dichloromethane was added and washed with aqueous sodium hydrogencarbonate and brine, dried over sodium sulphate, filtrated and concentrated in vacuo. The crude product was purified by flash chromatography (ethylacetate/heptane as eluent) to give 4-[(3,4-difluoro-phenyl)-propionyl-amino]-piperidine-1-carboxylic acid benzyl ester (10.5 g, 39%) as an yellow oil.

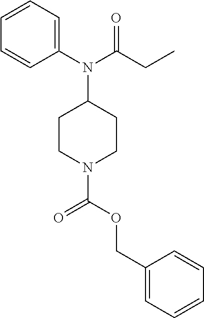

### 4-(Phenyl-propionyl-amino)-piperidine-1-carboxylic acid benzyl ester

Was prepared according to Example 5 from 4-phenylamino-piperidine-1-carboxylic acid benzyl ester and propionyl chloride.

### Example 6

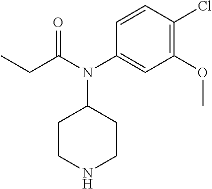

### N-(4-Chloro-3-methoxy-phenyl)-N-piperidin-4-yl-propionamide

N-(1-Benzyl-piperidin-4-yl)-N-(4-chloro-3-methoxy-phenyl)-propionamide (6.0 g, 15.5 mmol) was dissolved in dichloroethane (100 mL). 1-Chloroethyl chloroformate (8.53 mL, 77.5 mmol) was added and the reaction mixture was heated to reflux for 3 hours and then concentrated in vacuo. Methanol was added and the resulting mixture was heated to reflux for 2 hours followed by concentration in vacuo. Diethyl ether was added and the resulting solid was filtrated and dried. The solid material was dissolved in water, washed with diethyl ether, basified with sodium hydroxide (3 M). Extraction with diehylether, washing with brine, drying over sodium sulphate and concentration in vacuo gave N-(4-chloro-3-methoxy-phenyl)-N-piperidin-4-yl-propionamide 3.8 g (85%).

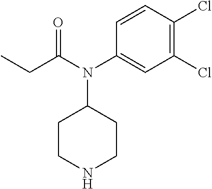

### N-(3,4-Dichloro-phenyl)-N-piperidin-4-yl-propionamide

Was prepared according to Example 6 from N-(1-benzyl-piperidin-4-yl)-N-(3,4-dichloro-phenyl)-propionamide.

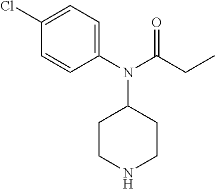

### N-(4-Chloro-phenyl)-N-piperidin-4-yl-propionamide

Was prepared according to Example 6 from N-(1-benzyl-piperidin-4-yl)-N-(4-chloro-phenyl)-propionamide.

### Example 7

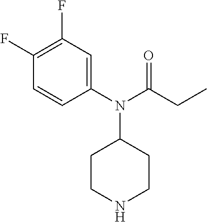

### N-(3,4-Difluoro-phenyl)-N-piperidin-4-yl-propionamide

4-[(3,4-Difluoro-phenyl)-propionyl-amino]-piperidine-1-carboxylic acid benzyl ester (10.5 g, 26.1 mmol) was dissolved in tetrahydrofuran (100 mL). A catalytic amount of palladium on carbon was added and the reaction was stirred under a hydrogen atmosphere for 4 hours at room temperature. Filtration through a pad of celite followed by evaporation in vacuo of the solvents gave N-(3,4-difluoro-phenyl)-N-piperidin-4-yl-propionamide (5.81 g, 83%) as a white solid.

### Example 8

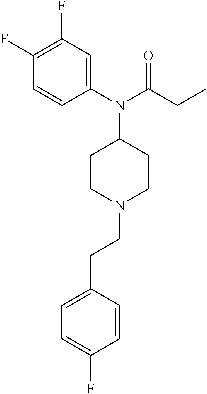

### N-(3,4-Difluoro-phenyl)-N-{1-[2-(4-fluoro-phenyl)-ethyl]-piperidin-4-yl}-propionamide, hydrochloric acid salt

N-(3,4-Difluoro-phenyl)-N-piperidin-4-yl-propionamide (500 mg, 1.86 mmol) 2-(4-fluorophenyl)ethylbromide (568 mg, 2.80 mmol) and diisopropylamine (0.39 mL, 2.80 mmol) were dissolved in N,N-dimethylformamid (10 mL) and heated to 60° C. for 4 hours. Diethylether (20 mL) was added and the organic phase was washed twice with aqueous sodium hydroxide, dried over sodium sulphate and filtrated. A solution of hydrochloric acid in ethanol was added and N-(3,4-difluoro-phenyl)-N-{1-[2-(4-fluoro-phenyl)-ethyl]-piperidin-4-yl}-propionamide precipitated as the hydrochloric acid salt (730 mg, 100%). LC-ESI-HRMS of [M+H]+ shows 391.1978 Da. Calc. 391.199722 Da, dev. −4.9 ppm

### N-(3,4-Difluoro-phenyl)-N-{1-[2-(4-methoxy-phenyl)-ethyl]-piperidin-4-yl}-propionamide, hydrochloric acid salt

Was prepared according to Example 8 from N-(3,4-Difluoro-phenyl)-N-piperidin-4-yl-propionamide and 1-(2-chloroethyl)-4-methoxybenzene. LC-ESI-HRMS of 20 [M+H]+ shows 403.2216 Da. Calc. 403.219709 Da, dev. 4.7 ppm.

### N-(3,4-Difluoro-phenyl)-N-{1-[2-(3-fluoro-phenyl)-ethyl]-piperidin-4-yl}-propionamide, hydrochloric acid salt

Was prepared according to Example 8 from N-(3,4-Difluoro-phenyl)-N-piperidin-4-yl-propionamide and 3-fluorophenethylbromide. LC-ESI-HRMS of [M+H]+ shows 391.2007 Da. Calc. 391.199722 Da, dev. 2.5 ppm.

### N-(4-Chloro-phenyl)-N-{1-[2-(4-methoxy-phenyl)-ethyl]-piperidin-4-yl}-propionamide, hydrochloric acid salt

Was prepared according to Example 8 from N-(4-chloro-phenyl)-N-piperidin-4-yl-propionamide and 1-(2-chloroethyl)-4-methoxybenzene. LC-ESI-HRMS of [M+H]+ shows 401.1992 Da. Calc. 401.199581 Da, dev. −0.9 ppm

### N-(4-Chloro-phenyl)-N-{1-[2-(4-fluoro-phenyl)-ethyl]-piperidin-4-yl}-propionamide, hydrochloric acid salt

Was prepared according to Example 8 from N-(4-chloro-phenyl)-N-piperidin-4-yl-propionamide and 2-(4-fluorophenyl)ethylbromide. LC-ESI-HRMS of [M+H]+ shows 389.178 Da. Calc. 389.179594 Da, dev. −4.1 ppm

### N-(4-Chloro-phenyl)-N-{1-[2-(3-fluoro-phenyl)-ethyl]-piperidin-4-yl}-propionamide, hydrochloric acid salt

Was prepared according to Example 8 from N-(4-chloro-phenyl)-N-piperidin-4-yl-propionamide and 3-fluorophenethylbromide. LC-ESI-HRMS of [M+H]+ shows 389.179 Da. Calc. 389.179594 Da, dev. −1.5 ppm.

### N-(3,4-Dichloro-phenyl)-N-{1-[2-(4-methoxy-phenyl)-ethyl]-piperidin-4-yl}-propionamide, hydrochloric acid salt

Was prepared according to Example 8 from N-(3,4-dichloro-phenyl)-N-piperidin-4-yl-propionamide and 1-(2-chloroethyl)-4-methoxybenzene. LC-ESI-HRMS of [M+H]+ shows 435.1624 Da. Calc. 435.160609 Da, dev. 4.1 ppm.

### N-(3,4-Dichloro-phenyl)-N-{1-[2-(3-fluoro-phenyl)-ethyl]-piperidin-4-yl}-propionamide, hydrochloric acid salt

Was prepared according to Example 8 from N-(3,4-dichloro-phenyl)-N-piperidin-4-yl-propionamide and 3-fluorophenethylbromide. LC-ESI-HRMS of [M+H]+ shows 423.1391 Da. Calc. 423.140622 Da, dev. −3.6 ppm

### N-(3,4-Dichloro-phenyl)-N-{1-[2-(4-fluoro-phenyl)-ethyl]-piperidin-4-yl}-propionamide, hydrochloric acid salt

Was prepared according to Example 8 from N-(3,4-dichloro-phenyl)-N-piperidin-4-yl-propionamide and 2-(4-fluorophenyl)ethylbromide. LC-ESI-HRMS of [M+H]+ shows 423.1401 Da. Calc. 423.140622 Da, dev. −1.2 ppm

### N-(4-Chloro-3-methoxy-phenyl)-N-{1-[2-(4-fluoro-phenyl)-ethyl]-piperidin-4-yl}-propionamide, hydrochloric acid salt

Was prepared according to Example 8 from N-(4-chloro-3-methoxy-phenyl)-N-piperidin-4-yl-propionamide and 2-(4-fluorophenyl)ethylbromide. LC-ESI-HRMS of [M+H]+ shows 419.1921 Da. Calc. 419.190159 Da, dev. 4.6 ppm

### N-(4-Chloro-3-methoxy-phenyl)-N-{1-[2-(4-methoxy-phenyl)-ethyl]-piperidin-4-yl}-propionamide, hydrochloric acid salt

Was prepared according to Example 8 from N-(4-chloro-3-methoxy-phenyl)-N-piperidin-4-yl-propionamide and 1-(2-chloroethyl)-4-methoxybenzene. LC-ESI-HRMS of [M+H]+ shows 431.2118 Da. Calc. 431.210146 Da, dev. 3.8 ppm

### N-(4-Chloro-3-methoxy-phenyl)-N-{1-[2-(3-fluoro-phenyl)-ethyl]-piperidin-4-yl}-propionamide, hydrochloric acid salt

Was prepared according to Example 8 from N-(4-chloro-3-methoxy-phenyl)-N-piperidin-4-yl-propionamide and 3-fluorophenethylbromide. LC-ESI-HRMS of [M+H]+ shows 419.1892 Da. Calc. 419.190159 Da, dev. −2.3 ppm

### Test Example

### Binding Data

Compounds have been tested in a binding assay using human recombinant opiate μ receptors. The assay was conducted as previously described by Simonin F et al [Simonin F et al, Mol. Pharmacol., 46(6), 1015-21, 1994], Simonin F et al [Simonin F et al, Proc. Natl. Acad. Sci. USA, 92(15), 7006-10, 1995], and Wang J B et al [Wang J B et al, FEBS Lett., 348(1), 75-9, 1994],

The test results are presented in Table 1 below.

TABLE 1

| Compound | μ |
|---|---|
| N-(3,4-Difluoro-phenyl)-N-{1-[2-(4-fluoro- | 91% inhib at 10 μM |
| phenyl)-ethyl]-piperidin-4-yl}-propionamide | |
| N-(3,4-Dichloro-phenyl)-N-{1-[2-(3-fluoro- | 88% inhib at 10 μM |
| phenyl)-ethyl]-piperidin-4-yl}-propionamide | |

### In Vitro Inhibition Activity

A number of compounds were tested for their ability to inhibit the reuptake of the monoamine neurotransmitters dopamine (DA) noradrenaline (NA) and serotonine (5-HT) in synaptosomes as described in WO 97/16451.

The test values are given as IC₅₀ (the concentration (μM) of the test substance which inhibits the specific binding of ³H-DA, ³H-NA, or ³H-5-HT by 50%).

Test results obtained by testing selected compounds of the present invention appear from the below table:

TABLE 2

| | 5-HT- | DA- | NA- |
|---|---|---|---|
| | uptake | uptake | uptake |
| Test compound | IC₅₀ (μM) | IC₅₀ (μM) | IC₅₀ (μM) |
| N-(3,4-Difluoro-phenyl)-N-{1-[2-(4- | 4.1 | 11 | 4.2 |
| fluoro-phenyl)-ethyl]-piperidin-4-yl}- | | | |
| propionamide | | | |
| N-(3,4-Dichloro-phenyl)-N-{1-[2-(3- | 1.8 | 5.6 | 0.56 |
| fluoro-phenyl)-ethyl]-piperidin-4-yl}- | | | |
| propionamide | | | |

1-11. (canceled)

12. A compound of Formula I: 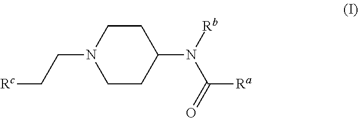
any of its stereoisomers or any mixture of its stereoisomers, or a pharmaceutically acceptable salt thereof; wherein
R^{a} represents hydrogen or alkyl;
which alkyl is optionally substituted with one or more substituents independently selected from the group consisting of:
halo, trifluoromethyl, trifluoromethoxy, cyano and alkoxy;
R^{b} and R^{c} independent of each other represent an aryl group;
which aryl group is substituted with one or more substituents independently selected from the group consisting of:
halo, trifluoromethyl, trifluoromethoxy, cyano and alkoxy.

13. The compound according to claim 12, any of its stereoisomers or any mixture of its stereoisomers, or a pharmaceutically acceptable salt thereof, wherein R^{a} represents alkyl.

14. The compound according to claim 12, any of its stereoisomers or any mixture of its stereoisomers, or a pharmaceutically acceptable salt thereof, wherein R^{b} represents a substituted phenyl.

15. The compound according to claim 12, any of its stereoisomers or any mixture of its stereoisomers, or a pharmaceutically acceptable salt thereof, wherein R^{c} represents a substituted phenyl.

16. The compound of claim 12 which is
N-(3,4-Difluoro-phenyl)-N-{1-[2-(4-fluoro-phenyl)-ethyl]-piperidin-4-yl}-propionamide;
N-(3,4-Difluoro-phenyl)-N-{1-[2-(4-methoxy-phenyl)-ethyl]-piperidin-4-yl}-propionamide;
N-(3,4-Difluoro-phenyl)-N-{1-[2-(3-fluoro-phenyl)-ethyl]-piperidin-4-yl}-propionamide;
N-(4-Chloro-phenyl)-N-{1-[2-(4-methoxy-phenyl)-ethyl]-piperidin-4-yl}-propionamide;
N-(4-Chloro-phenyl)-N-{1-[2-(4-fluoro-phenyl)-ethyl]-piperidin-4-yl}-propionamide;
N-(4-Chloro-phenyl)-N-{1-[2-(3-fluoro-phenyl)-ethyl]-piperidin-4-yl}-propionamide;
N-(3,4-Dichloro-phenyl)-N-{1-[2-(4-methoxy-phenyl)-ethyl]-piperidin-4-yl}-propionamide;
N-(3,4-Dichloro-phenyl)-N-{1-[2-(3-fluoro-phenyl)-ethyl]-piperidin-4-yl}-propionamide;
N-(3,4-Dichloro-phenyl)-N-{1-[2-(4-fluoro-phenyl)-ethyl]-piperidin-4-yl}-propionamide;
N-(4-Chloro-3-methoxy-phenyl)-N-{1-[2-(4-fluoro-phenyl)-ethyl]-piperidin-4-yl}-propionamide;
N-(4-Chloro-3-methoxy-phenyl)-N-{1-[2-(4-methoxy-phenyl)-ethyl]-piperidin-4-yl}-propionamide;
N-(4-Chloro-3-methoxy-phenyl)-N-{1-[2-(3-fluoro-phenyl)-ethyl]-piperidin-4-yl}-propionamide;
any of its stereoisomers or any mixture of its stereoisomers, or a pharmaceutically acceptable salt thereof.

17. A pharmaceutical composition, comprising a therapeutically effective amount of a compound of claim 12, any of its stereoisomers or any mixture of its stereoisomers, or a pharmaceutically acceptable salt thereof, together with at least one pharmaceutically acceptable carrier, excipient or diluent.

18. A method for treatment, prevention or alleviation of a disease or a disorder or a condition of a living animal body, including a human, which disorder, disease or condition is responsive to responsive to modulation of the opioid receptor, which method comprises the step of administering to such a living animal body in need thereof a therapeutically effective amount of a compound according to claim 12, any of its stereoisomers or any mixture of its stereoisomers, or a pharmaceutically acceptable salt thereof.

19. The method according to claim 18, wherein the disease, disorder or condition responsive to modulation of the opioid receptor is pain, postoperative pain, chronic pain, cancer pain, neuropathic pain, pain during labour and delivery, migraine, drug addiction, heroin addiction, cocaine addiction, alcoholism, irritable bowel syndrome, constipation, nausea, vomiting, pruritic dermatoses, allergic dermatitis, atopy, eating disorders, opiate overdoses, depression, smoking, sexual dysfunction, shock, stroke, spinal damage, head trauma, diarrhoea, urinary incontinence and inflammatory reactions.
